(19) United States
(12) Patent Application Publication
(10) Pub. No.: US 2016/0152117 A1
(43) Pub. Date: Jun. 2, 2016

(54) ARRANGEMENT OF A FRAGRANCE DISPENSER IN THE CABIN OF A MOTOR VEHICLE

(71) Applicant: GM GLOBAL TECHNOLOGY OPERATIONS LLC, Detroit, MI (US)

(72) Inventors: Fredrik BACKMAN, Frankfurt am Main (DE); Valter SKIBINSKI, Wallertheim (BR); Jun Hyuck EOH, Russelsheim (DE)

(73) Assignee: GM GLOBAL TECHNOLOGY OPERATIONS LLC, Detroit, MI (US)

(21) Appl. No.: 14/949,943

(22) Filed: Nov. 24, 2015

(30) Foreign Application Priority Data
Nov. 27, 2014 (DE) 202014009431.4

(51) Int. Cl. B60H 3/00; A61L 9/03

(52) U.S. Cl. CPC B60H 3/0007; A61L 9/03; A61L 9/035

(57) ABSTRACT

A fragrance dispenser is provided to be disposed in the cabin of a motor vehicle, while maintaining a structurally simple configuration and uncomplicated mountability for the dispenser. The dispenser is to ensure an efficient improvement of the fragrance equipment in the cabin of the motor vehicle. The fragrance dispenser may be disposed in the cabin of a motor vehicle integral or arranged with interior trim that detachably accommodates the dispenser. The dispenser may have a housing for accommodating the fragrances, and the housing may further have at least one opening for dispensing the fragrances from the housing. The fragrance dispenser may also have an electric heater for heating the fragrances located in the housing.

### CROSS-REFERENCE TO RELATED APPLICATION

This application claims priority to German Patent Application No. 202014009431.4, filed Nov. 27, 2014, which is incorporated herein by reference in its entirety.

### TECHNICAL FIELD

The present disclosure pertains to an arrangement of a fragrance dispenser in the cabin of a motor vehicle. In particular, the present disclosure relates to an arrangement for use in a passenger car.

### BACKGROUND

Depending on personal sensitivities or standards, vehicle passengers want fragrance equipment in the vehicle cabin which they find comfortable. This is most easily accomplished by incorporating aromatic sticks that generally exhibit a special design inside the cabin of the motor vehicle. In particular, these aromatic sticks are hung from a rear view mirror or sun visor mount.

### SUMMARY

In accordance with herein described embodiments of the invention, a fragrance dispenser is provided to be disposed in the cabin of a motor vehicle, while maintaining a structurally simple configuration and uncomplicated mountability for the dispenser. The dispenser is to ensure an efficient improvement of the fragrance equipment in the cabin of the motor vehicle.

In accordance with embodiments of the the invention, an arrangement of a fragrance dispenser to be disposed in the cabin of a motor vehicle includes an interior trim that has a holding device for detachably accommodating the dispenser. The dispenser has a housing for accommodating the fragrances, and the housing has at least one opening for dispensing the fragrances from the housing, along with an electric heater for heating the fragrances located in the housing. When the dispenser is connected with the holding device, the electrical contacts of the holding device and heater are connected.

As a consequence, the dispenser may be detachably connected with the holding device in the arrangement according to the embodiments of the invention. This makes it possible to design the dispenser as an accessory. All that need be done is to provide a holding device for the dispenser in the cabin of the motor vehicle. This holding device may also serve to accommodate other functional elements. The arrangement of the dispenser may also provide an electric heater for heating the fragrances. The ability to heat the fragrances results in an especially efficient release of the fragrances, and hence, for efficiently improving the fragrance equipment in the cabin of the motor vehicle. Power is supplied to the electric heater via the holding, device. When the dispenser is connected with the holding device, the electrical contacts of the holding device and heater are connected. If the electric heater is turned on, the fragrances are heated, and dispensed through the at least one opening in the housing in the cabin of the motor vehicle located outside of the housing

In further embodiments of the invention, the dispenser may include a light unit for illuminating the interior trim, wherein the electrical contacts of the holding device and light unit are connected when the dispenser is connected with the holding device. As a consequence, the dispenser is used not just to dispense fragrances, but further to illuminate the interior trim. In this way, the area of the interior trim or cabin adjacent to the dispenser can be illuminated on the one hand, and an especially positive lighting impression can be imparted to this adjacent area of the dispenser on the other. It is especially advantageous if the light unit can be operated with different colored lights.

In particular embodiments, the interior trim involves a center console or dashboard of the motor vehicle. Partial areas normally remain unused in the vicinity of the center console or dashboard, and can be easily utilized by attaching the holding device and electrically contacting the holding device for accommodating the dispenser.

The housing that serves to accommodate the fragrances may further have several openings. In particular, the openings are part of a ventilation grate of the housing. This configuration yields a rather large opening surface in the housing given an optically advantageous design. These ventilation grates allow enough fragrance to be dispensed to the cabin that envelops the housing.

A switch for switching the heater and/or light unit may be integrated into the housing. In particular, this switch may be a capacitive touch-sensitive switch. The latter can be easily and ergonomically favorably operated, while keeping the housing and switch arranged in an aesthetically advantageous configuration.

The light unit may be situated in the area of a lower side of a housing. This makes it possible to illuminate an adjacent area of the dispenser for the passengers without any glare. An advantageous configuration of the light unit includes at least one LED and/or a fiber optic device extending in the longitudinal direction of the housing to guide the light emitted by the LED. This configuration also results in a sufficient, antiglare illumination of the cabin area adjacent to the housing over the entire length of the housing.

The light unit may have several different kinds of LEDs for feeding light into the fiber optic device, in particular for feeding a respective colored light into the fiber optic device.

In order to easily and detachably join the dispenser and holding device, the dispenser may be joined with the holding device by a clip-on connector. As a result, the dispenser can be easily plugged onto the holding device, and pulled off the latter for detachment purposes.

In particular embodiments, the holding device may have a cross section shaped like a dovetail. In this case, the dispenser engages an upper projection of the dovetail from behind while in the position joined with the holding device, and, in the area of a lower projection of the dovetail, the dispenser engages this lower projection from behind by an elastic holding element.

Particular embodiments of the invention provide dispensers having various fragrances and/or various colored lights being joined with the holding device. This leaves it largely at the discretion of the passenger(s) to select whatever fragrances or colored lights are used. The ability to detachably connect the dispenser and holding device enables the uncomplicated replacement of dispensers.

### BRIEF DESCRIPTION OF THE DRAWINGS

The present invention will hereinafter be described in conjunction with the following drawing figures, wherein like numerals denote like elements.

FIG. 1 is a partial section of a center console of a passenger car with the fragrance dispenser attached to the center console;

FIG. 2 is the arrangement according to FIG. 1, with the dispenser detached from the center console;

FIG. 3 is a vertical section through the center console, a holding device allocated thereto, and a dispenser joined with the holding device based on the functional layout according to FIG. 1;

FIG. 4 is an essentially front view of the dispenser in the area of a front housing part of the dispenser;

FIG. 5 is an essentially rear view of the dispenser with the rear housing part of the dispenser removed.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description.

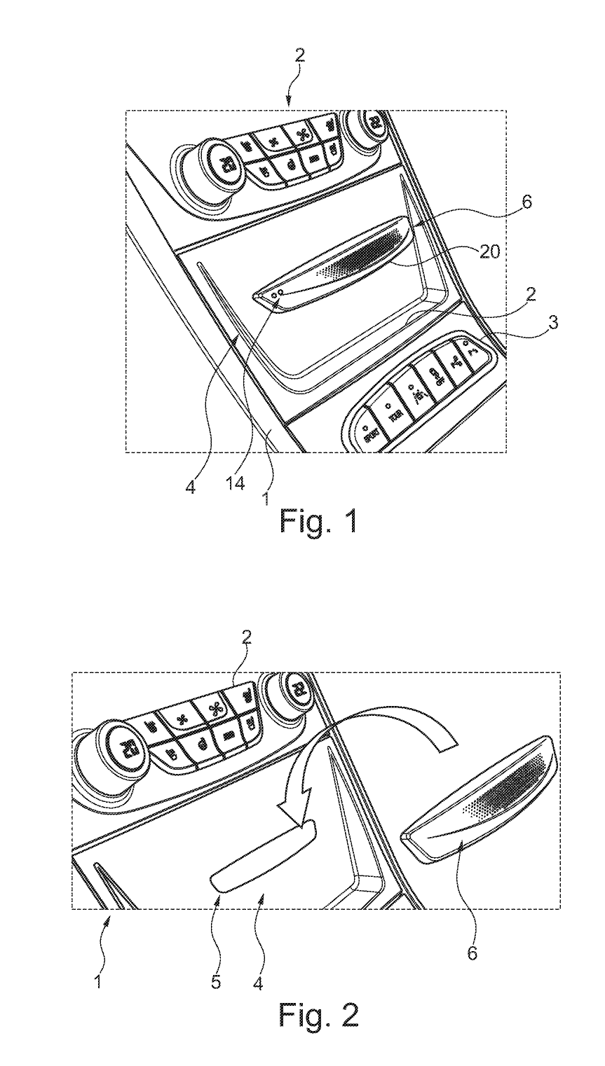

FIGS. 1 and 2 illustrate an interior trim 1 designed as the center console of a passenger car for a partial area of the center console in which the latter rises to form a dashboard of the passenger car (not shown). Present between the upper control elements 2 and lower control elements 3 is a flat intermediate area 4, to which a holding device 5 is attached. The latter is oblong in design and horizontally oriented. The holding device 5 is used to detachably, specifically accommodate a fragrance dispenser 6.

FIG. 1 shows the dispenser 6 connected with the holding device 5, while FIG. 2 shows the dispenser 6 before attached to the holding device 5.

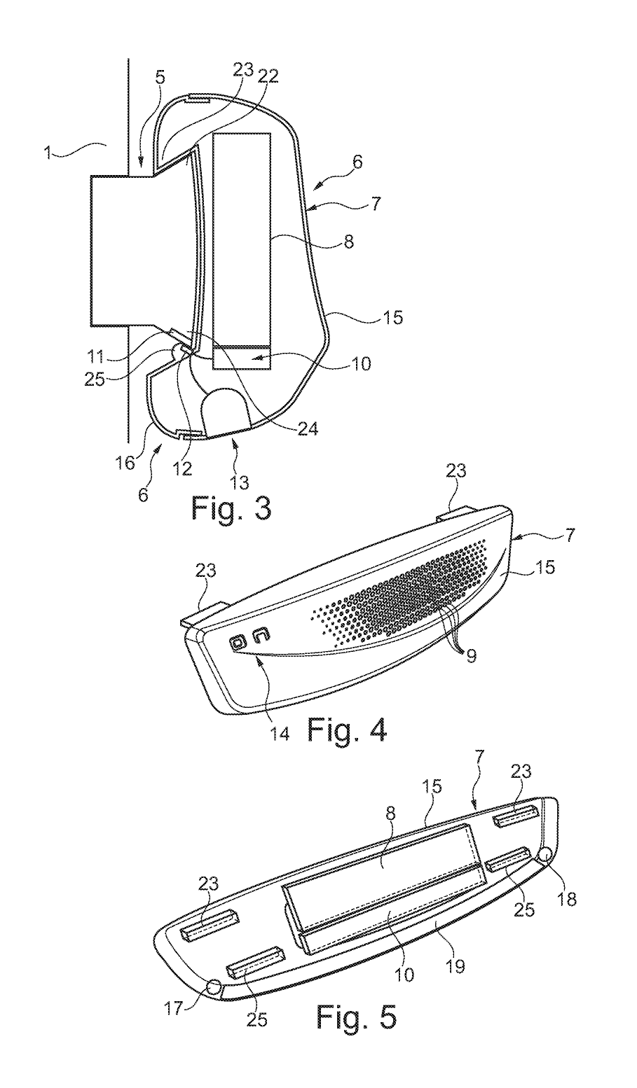

FIGS. 3 to 5 provide a further, detailed illustration depicting the arrangement of the holding device 5 and dispenser 6 or the dispenser 6 as such:

The dispenser 6 exhibits housing 7 for accommodating the fragrances. These fragrances are contained in a cushion 8, receptacle or the like, which is situated inside of the housing 7 and fixed in place therein. The housing 7 has a plurality of openings 9, which form a ventilation grate, for dispensing the fragrances from the housing 7. Arranged below the cushion 8 is an electric heater 10 for heating the fragrances located in the housing 7, specifically located in the cushion 8. When the dispenser 6 is connected with the holding device 5 as illustrated on FIG. 3, the electrical contacts 11, 12 of the holding device 5 and heater 10 are connected.

The dispenser 6 further has a light unit 13 for illuminating the interior trim 1 or center console. The electrical contact 12 allocated to the housing 7 here serves not just to supply power to the electric heater 10, but also to supply power to the light unit 13 via a separate line. As a consequence, if the heater 10 is energized, the light unit 13 is also energized. The housing 7 integrates a switch 14, which is used to electrically switch the heater 10 and light unit 13. This switch 14 is designed as a capacitive touch-sensitive switch.

The housing 7 consists of two housing parts 15 and 16, which are joined together by, for example, clip connectors. The electrical contact 12 is situated in the area of the interior housing part 15 facing the interior trim 1 or center console, while the light unit 13 along with the heater 10 and cushion 8 are arranged in the area of the other, exterior housing part 16 facing the passengers.

The light unit 13 is situated in the area of the lower side of the housing 7. The light unit 13 may have two different-colored LEDs 17, 18 for feeding light into a fiber optic device 19, which extends in the longitudinal direction of the housing 7. If the LED 17 is turned on, the latter feeds blue light into the fiber optic device 19, so that the dispenser 6 appears with a blue contour 20 in the area of its lower edge, and a bulge 21 of the interior trim 1 or central console arranged under the dispenser 6 is otherwise illuminated in blue by the fiber optic device 19. Turning the red LED 18 on instead yields a red contour 20 and a bulge 21 illuminated in red.

In order to easily attach the dispenser 6 to the holding device 5, the holding device 5 has a cross section shaped like a dovetail. The dispenser 6, in the position joined with the holding device 5, engages an upper projection 22 of the dovetail of the holding device 5 from behind via a projection 23 with its housing part 15, and, in the area of a lower projection 24 of the dovetail of the holding device 5, engages this lower projection 24 from behind by means of an elastic holding element 25. In this way, the dispenser 6 can be easily mounted to the holding device 5 or demounted from the latter.

FIG. 4 illustrates projections 24 arranged in partial areas of the housing 7. The positions of the projections 23 forming the interior housing part 16 and elastic holding elements are also delineated in the rear view of the exterior housing part 15.

The configuration of the described arrangement according to embodiments of the invention makes it possible to connect dispensers exhibiting various fragrances and/or different colored lights with the holding device.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment, it being understood that various changes may be made in the function and arrangement of elements described in an exemplary embodiment without departing from the scope of the invention as set forth in the appended claims and their legal equivalents.

1-14. (canceled)

15. An arrangement of a fragrance dispenser in the cabin of a motor vehicle, with an interior trim, including a holding device for detachably accommodating the dispenser, wherein the dispenser includes a housing for accommodating the fragrances, and the housing has at least one opening for dispensing the fragrances from the housing, an electric heater for heating the fragrances located in the housing, and wherein, when the dispenser is connected with the holding device, the electrical contacts of the holding device and heater are connected.

16. The arrangement according to claim 15, wherein the dispenser includes a light unit configured to illuminate the interior trim.

17. The arrangement according to claim 15 wherein the interior trim is a center console or dashboard.

18. The arrangement according to claim 15, wherein the housing includes se openings for dispensing the fragrances from the housing, the openings being part of a. ventilation grate of the housing.

19. The arrangement according to claim 16, comprising a switch for switching the heater or the light unit is integrated Ito the housing.

20. The arrangement according to claim 19, wherein the switch is a capacitive touch-sensitive switch.

21. The arrangement according to claim 16, wherein the light unit is disposed in lower side of the housing.

22. The arrangement according to claim 16, wherein the light unit includes at least one LED and a fiber optic device extending in a longitudinal direction of the housing to guide the light emitted by the LED.

23. The arrangement according to claim 22, wherein the light unit includes several different-colored LEDs coupled to the fiber optic device.

24. The arrangement according to claim 15, wherein the dispenser is oblong in design, and, in a horizontal configuration, can be connected with the holding device.

25. The arrangement according to claim 15, wherein the dispenser is configured to be joined with the holding device by a clip connector.

26. The arrangement according to claim 15, wherein the holding device has a cross section shaped like a dovetail.

27. The arrangement according to claim 26, wherein the dispenser, in the position joined with the holding device, engages an upper projection of the dovetail of the holding device from behind, and, in the area of a lower projection of the dovetail of the holding device, an elastic holding element engages the lower projection.

28. The arrangement according to claim 15, wherein the dispensers have more than one fragrance.

29. The arrangement according to claim 15, where the dispensers have more than one colored LED.
